(19) United States
(12) Patent Application Publication
(10) Pub. No.: US 2006/0100489 A1
(43) Pub. Date: May 11, 2006

(54) Method and apparatus for determining tissue viability

(75) Inventors: Benny Pesach, Rosh-Ha'ayin (IL); Michal Balberg, Jerusalem (IL)

Correspondence Address: WOLF, BLOCK, SCHORR & SOLIS-COHEN LLP, 250 PARK AVENUE, NEW YORK, NY, 10177, US

(73) Assignee: GLUCON, INC., BOULDER, CO (US)

(21) Appl. No.: 10/519,024

(22) PCT Filed: Jun. 25, 2003
(86) PCT No.: PCT/IL03/00533

§ 371 Date: Nov. 30, 2005

Related U.S. Application Data
(60) Provisional application No. 60/391,038, filed on Jun. 25, 2002.

(51) Int. Cl. A61B 5/00

(52) U.S. Cl. 600/310

(57) ABSTRACT

A tissue viability monitor (TVM) for determining viability of a biological tissue comprising: at least one light source controllable to illuminate the tissue with light that generates photoacoustic waves therein; at least one acoustic transducer that generates signals responsive to the photoacoustic waves; and a controller that receives the signals and processes the signals to determine at least one characteristic of the tissue and a measure of viability responsive to the determined at least one characteristic.

### RELATED APPLICATION

This application claims the benefit under 119(e) of 60/391,038 filed Jun. 25, 2002, the disclosure of which is incorporated herein by reference.

### FIELD OF THE INVENTION

The invention relates to methods and apparatus for determining if biological tissue is viable and in particular to identifying and locating viability compromised tissue in a body.

### BACKGROUND OF THE INVENTION

Determining viability of tissue in an organ or a region of an organ, or an aspect of viability such as an amount of blood flow to the organ or region thereof, is often an advantageous or necessary adjunct of therapeutic and diagnostic procedures. For example, monitoring success of a transplant in integrating with a body or tissue into which it is transplanted requires monitoring viability of the transplant. Determining where to drill holes in the heart of a patient undergoing myocardial revascularization requires identifying ischemic regions of heart tissue and, advantageously, a degree of ischemia suffered by the regions. It has also been recognized that tissue can exhibit different degrees of viability and biological tissue is not necessarily either completely viable or necrotic but may exhibit intermediate states of viability. For example, heart tissue may appear necrotic but actually be in a state of “hibernation”. Properly identifying and locating tissue in a state of hibernation can aid in determining a type of therapy to be used in repairing and reviving the hibernating tissue. To an extent that methods and apparatus for determining tissue viability accurately identify different states of impaired viability and locus of viability-compromised tissue, the methods and apparatus provide for improved diagnosis and therapy. Hereinafter, viability of tissue and aspects of its viability, such as magnitude of blood flow to the tissue and oxygen uptake and utilization, are collectively referred to as viability.

Among methods used for assessing tissue viability are visual inspection, imaging methods such as PET, MRI and ultrasound imaging, Thallium perfusion, and near infrared (NIR) spectroscopic assaying of tissue analytes whose concentrations, or changes therein, are useable as indicators of viability.

PET and MRI imaging methods while useable to provide relatively accurate assessment of location and degree of viability require large and expensive equipment, are not readily available and cannot be conveniently used to provide rapid tissue diagnosis in an emergency or during an operation. Thallium perfusion methods also generally require large and expensive equipment and are time consuming. In addition, Thallium perfusion methods have proven relatively frequently to be unreliable. Ultrasound imaging techniques are relatively insensitive to differences in viability of tissue regions and as a result generally provide relatively poor spatial resolution for distinguishing between tissue regions having different degrees of viability. Whereas NIR spectroscopic methods are relatively inexpensive and apparatus for practicing the methods can be packaged in catheters, the methods do not generally provide accurate localization of compromised tissue. Scattering of light used in NIR spectroscopy, can be substantial, reduces accuracy of NIR measurements and mitigates against extracting accurate position information from NIR spectroscopy signals as to which tissue voxels absorb or reflect the light. In particular NIR light is relatively strongly scattered by outer tissue layers of the body. To reduce scattering effects on NIR spectroscopy “viability” signals, NIR light used in viability measurements of tissue is generally required to traverse a relatively long optical path through the tissue before intensity of the light is measured to determine an absorption and/or scattering coefficient for the light. However, the relatively long optical path attenuates amplitude of the signals and tends to decrease signal to noise.

U.S. Pat. No. 4,281,645 describes using NIR spectroscopy to assay the redox state of the enzyme cytochrome a,a₃ as a measure of oxygen sufficiency in an organ. The assay is performed by transmitting light at a wavelength of about 840 nm through the body from a first side to a second side of the body along an optic path that passes through the organ. Intensity of the light is measured at the second side to determine absorption of the light along the path and therefrom a measure of the concentration of redox cytochrome a,a₃ in the organ. An assay of hemoglobin and oxyhemoglobin in the organ is performed by measuring absorption of light at NIR wavelengths of 760 nm and 815 nm along the optic path. Localization of a source of absorption of the light to a particular region along the path is not available from the measurement. For localization, the inventor states that known techniques of axial tomography are available. FIG. 10 in the patent illustrates a “tomography-like technique” and FIG. 11 “is a schematic diagram of an axial tomography system according to the invention”.

However, it appears that localization methods suggested in U.S. Pat. No. 4,281,645 are not sufficiently satisfactory. U.S. Pat. No. 4,223,680, subsequent to and to the same inventor as the inventor of U.S. Pat. No. 4,281,645, describes assaying the same analytes discussed in the U.S. Pat. No. 4,281,645 patent by measuring reflection of light by organs in the body at the above noted wavelengths. The U.S. Pat. No. 4,223,680 patent notes that the reflection method “should be expected for many applications to provide better localization of the area from which signals are obtained”.

U.S. Pat. No. 5,497,770 describes monitoring tissue viability by diffusing into the tissue basic ingredients needed for cellular respiration and resulting energy production (oxygen, glucose, low energy phosphates) to stimulate tissue activity. The result of the activity is detected by performing measurements of substance uptake, oxygen utilization and/or oxidation-reduction (redox) stores of respiratory enzymes. In an embodiment of the invention NIR spectroscopy is used to perform the measurements. Apparatus for monitoring tissue viability in accordance with the patent may be configured in a catheter and the patent notes that a useful catheter configuration for analyte detection using NIR spectroscopy is described in U.S. Pat. Nos. 5,161,531 and 5,127,409.

U.S. Pat. No. 5,813,403 uses NIR spectroscopy to determine pH of tissue being examined to assess viability. Lactic acid and hydrogen are by products of anaerobic metabolism and accumulate in tissue that is compromised by insufficient circulation. As a result, pH can be used as a measure of blood flow, blood flow history and ischemia. NIR reflection spectra are used to determine tissue pH.

U.S. Pat. No. 6,277,082 B1 describes a method of detecting “ischemic biological tissue by temporarily altering the temperature of the tissue and then monitoring the thermal profile of the tissue as it returns to normal temperature. Tissue areas of slower response time correspond to areas of reduced blood flow (ischemia).” An ischemia detection device for practicing the method comprises a catheter having a distal end that is placed adjacent to tissue to be tested for ischemia. The distal end has a “temperature alteration mechanism configured to alter temperature of a finite section of tissue” and a temperature detector for monitoring the thermal profile. The temperature alteration mechanism alters the temperature by delivering to the finite section of tissue a cooled or heated liquid or by heating the finite section of tissue with an electrical current. In an embodiment of the invention the temperature detector comprises an optic fiber located in the catheter such that an optic end of the fiber is positioned in the distal end of the catheter. The optic end receives IR light from the finite section of tissue and transmits the light to an IR detector that creates a thermal image of the tissue section.

The disclosures of all the U.S Patents referenced above are incorporated herein by reference.

There is a need for inexpensive apparatus and methods that can perform viability tests of tissue rapidly and provide improved spatial resolution of regions of tissue having different degrees of viability.

### SUMMARY OF THE INVENTION

An aspect of some embodiments of the present invention relates to providing improved apparatus, hereinafter a “tissue viability monitor (TVM)”, and methods for measuring tissue viability.

An aspect of some embodiments of the present invention relates to providing a TVM and methods that can relatively accurately determine location of tissue having impaired viability.

An aspect of some embodiments of the present invention relates to providing a TVM for performing a plurality of different viability tests on a region of tissue to determine viability of the tissue.

In accordance with an embodiment of the present invention, a TVM for assessing viability of tissue comprises a light source, which illuminates the tissue with light that generates photoacoustic waves therein, and at least one acoustic transducer that generates signals responsive to the photoacoustic waves. The signals are transmitted to a controller that processes the signals to determine a characteristic of the tissue and a measure of viability responsive to the determined characteristic. In accordance with an embodiment of the present invention, the signals are processed to determine locations of sources of the photoacoustic waves. The locations of the sources are associated with viability measurements based on photoacoustic waves that respectively originate from the sources to provide measurements of viability as a function of location. In accordance with an embodiment of the present invention, the characteristic is an absolute or relative concentration of an analyte, such as for example cytochrome a,a₃ or Hydrogen ion concentration (i.e. pH), in the tissue and/or a spatial or temporal change in the concentration that can be used to indicate viability. In accordance with an embodiment of the present invention, the light source illuminates the tissue with at least one pulse of light that is absorbed by the analyte. Signals generated by the acoustic detector responsive to photoacoustic waves stimulated by light absorbed by the analyte from the at least one pulse are processed by the controller to determine concentration and/or change in concentration of the analyte. Any of various methods known in the art, or methods described in PCT Publication WO 02/15776, the disclosure of which is incorporated herein by reference, may be used to determine concentration or change therein of the analyte from the photoacoustic signals. The concentration and/or change therein is used to estimate viability in accordance with any appropriate method known in the art.

Photoacoustic waves stimulated by the absorbed light that are incident on the at least one transducer arrive at the transducer at times that are functions of locations of their respective sources in the illuminated tissue at which they are generated. In accordance with an embodiment of the present invention, signals produced by the at least one acoustic transducer responsive to the incident photoacoustic waves are processed to determine spatial coordinates of the sources. The locations of the sources of the photoacoustic waves are used to determine concentration of the analyte and/or change therein and therefrom tissue viability, as a function of spatial location. As a result, for example, viability of tissue beneath a surface of an organ can be determined, in accordance with an embodiment of the present invention, as a function of depth below the surface as well as lateral position relative to the surface.

Sources of photoacoustic waves can be located using methods known in the art to a relatively high degree of accuracy. Location of tissue interfaces at which changes in optical absorption characteristics generated by differences in concentration of an analyte can often be determined using the photoacoustic effect to within 10 micrometers. As a result, a TVM, in accordance with an embodiment of the present invention, can be used to locate and accurately “map” volumes regions in the illuminated tissue having different degrees of viability and therefore different concentrations of an analyte whose concentration is indicative of viability. In particular, for example, a TVM, in accordance with an embodiment of the present invention, may be used to detect and accurately locate viability-compromised tissue, such as ischemic tissue. A TVM, in accordance with an embodiment of the present invention, therefore provides substantial advantages relative to conventional NIR spectroscopy apparatus for determining viability by providing enhanced capability to spatially locate viability-compromised tissue.

It is further noted that photoacoustic waves are attenuated as a function of propagation path length in biological tissue at a rate that is generally less than a typical attenuation rate of NIR light waves in biological tissue. As a result, a range for detecting reaction of a tissue voxel to illumination by NIR light is generally greater if the reaction is determined responsive to photoacoustic waves received from the voxel rather than responsive to NIR light received from the voxel. Alternatively, for a given distance of the voxel from a detector, signal to noise is generally greater for measurements of the voxel reaction to NIR illumination if the measurements are determined using photoacoustic waves received from the voxel rather than NIR light received from the voxel. A TVM, in accordance with an embodiment of the present invention, therefore generally provides an improved diagnosis range and/or signal to noise than conventional prior art devices that use NIR spectroscopy for determining viability.

In some embodiments of the present invention, the characteristic of the tissue that is used to determine viability is a “temperature” relaxation time of the tissue that describes the way a difference in temperature between the tissue and an ambient tissue temperature relaxes to zero. Similarly to the temperature relaxation method described in U.S. Pat. No. 6,277,082 referenced above, a temperature difference is generated between the tissue and an ambient temperature of surrounding tissue. The temperature of the tissue is measured thereafter and a time it takes for the temperature difference to relax to zero is determined and used to estimate viability.

However, unlike in U.S. Pat. No. 6,277,082, in accordance with an embodiment of the present invention, temperature of the tissue is measured using the photoacoustic effect. Optionally, measuring the temperature of the tissue is accomplished by “photoacoustically” measuring the temperature of water in the tissue in accordance with a method described in U.S. Provisional Application 60/331,408, the disclosure of which is incorporated herein by reference. By measuring temperature photoacoustically, in accordance with an embodiment of the present invention, temperature measurements of the tissue can be determined as a function of location within the tissue. As a result, accuracy of the method of determining viability by temperature relaxation time is improved and viability as a function of location in the tissue can be determined.

According to an aspect of some embodiments of the present invention, focussing acoustic energy on the tissue to heat the tissue generates the temperature difference. Optionally, the energy is focussed on the tissue from outside the body and therefore permits, unlike the methods described in U.S. Pat. No. 6,277,082, generating the temperature difference without contacting the tissue. Optionally, the at least one transducer comprises a phased array of acoustic transducers and energy is focussed on the tissue by the phased array. In such embodiments of the present invention, temperature relaxation assessment of viability may be performed without any invasive procedure.

In some embodiments of the present invention, a TVM performs a plurality of different types of measurements of viability on a region of tissue and uses the different measurements to determine tissue viability. For example a TVM, in accordance with an embodiment of the present invention, is optionally configured to perform at least two of an assay of cytochrome a,a_{3,} oxyhemoglobin pH and a determination of temperature relaxation to determine viability.

In some embodiments of the present invention, components of a TVM are mounted in a catheter suitable for percutaneous introduction into a patient's body and the TVM is used to diagnose viability of tissue in an organ of the patient percutaneously. The catheter has a “probe end” that is threaded through the patient's vascular system or through a suitable body orifice to be positioned in a neighborhood of tissue to be diagnosed. The tissue is illuminated by light transmitted from the probe end and, optionally, acoustic energy from photoacoustic waves generated responsive to the light is received by at least one acoustic transducer mounted in the probe end.

There is therefore provided in accordance with an embodiment of the present invention, a tissue viability monitor (TVM) for determining viability of a biological tissue comprising: at least one light source controllable to illuminate the tissue with light that generates photoacoustic waves therein; at least one acoustic transducer that generates signals responsive to the photoacoustic waves; and a controller that receives the signals and processes the signals to determine at least one characteristic of the tissue and a measure of viability responsive to the determined at least one characteristic.

Optionally, the controller processes the signals to determine locations of sources of the photoacoustic waves within the tissue. Optionally, the locations of sources of photoacoustic waves are determined with a resolution equal to or better than about 100 micrometers. Optionally, the locations of sources of photoacoustic waves are determined with a resolution equal to or better than about 50 micrometers. Optionally, the locations of sources of photoacoustic waves are determined with a resolution equal to or better than about 20 micrometers.

In some embodiments of the present invention, the at least one characteristic of the tissue comprises a concentration of at least one analyte. Optionally, the at least one analyte is a plurality of analytes. Additionally or alternatively, the at least one analyte comprises the redox state cytochrome a,a₃. In some embodiments of the present invention, the at least one analyte comprises Hydrogen ions. In some embodiments of the present invention, the at least one analyte comprises hemoglobin. In some embodiments of the present invention, the at least one analyte comprises oxygenated hemoglobin.

In some embodiments of the present invention, the TVM comprises a heat pump that the controller controls to generate a temperature difference between the tissue and an ambient temperature of surrounding tissue and wherein the at least one characteristic comprises a relaxation time characteristic of a time period during which the temperature difference relaxes to zero. Optionally, the heat pump comprises an acoustic transducer of the at least one acoustic transducer, which the controller controls to transmit acoustic waves to the tissue that generate the temperature difference. Additionally or alternatively, to determine the relaxation time the light source illuminates the tissue with light at a wavelength at which light is absorbed by water to generate photoacoustic waves in the tissue and the controller uses the signals generated by the at least one transducer to determine temperature of water in the tissue and thereby of the tissue.

In some embodiments of the present invention, the controller determines temperature of the tissue during generation of the temperature difference to monitor the generation of the temperature difference. Optionally, the controller controls the heat pump responsive to the determined temperature.

A TVM according to any of the preceding claims and comprising a catheter having a probe end that is positioned in a neighborhood of or in contact with the tissue to determine tissue viability and wherein the light source comprises an optic fiber having an optic end located at the probe end from which optic end light that illuminates the tissue is radiated. Optionally, the at least one at least one acoustic transducer comprises at least one acoustic transducer mounted in the probe end of the catheter.

There is further provided in accordance with an embodiment of the present invention, a tissue viability monitor (TVM) for determining viability of a biological tissue comprising: a heat pump controllable to non-invasively generate a temperature difference between the tissue and an ambient temperature of surrounding tissue; means for non-invasively determining a temperature of the tissue; and a controller that determines from the determined temperature a relaxation time characteristic of a time period during which the temperature difference relaxes to zero. Optionally, the heat pump comprises an acoustic transducer, which the controller controls to transmit acoustic waves to the tissue that generate the temperature difference. Additionally or alternatively, the means for non-invasively determining a temperature of the tissue comprises means for non-invasively determining a temperature of water in the tissue.

In some embodiments of the present invention, the means for determining a temperature of the water comprises: a light source controllable to illuminate the tissue with light which is absorbed by the water and generates photoacoustic waves in the tissue; at least one acoustic transducer that generates signals responsive to the photoacoustic waves; and a controller that receives the signals and processes the signals to determine the temperature of the water.

In some embodiments of the present invention, the means for determining a temperature of the water comprises: an acoustic transducer that transmits acoustic waves that are incident on the tissue; an acoustic transducer that generates signals responsive to acoustic waves scattered from the transmitted waves by the tissue; a controller that receives the signals and determines a characteristic of the scattered acoustic waves which it uses to determine temperature of the tissue. Optionally, the characteristic is a frequency shift of the scattered acoustic waves relative to a fundamental acoustic frequency of the structure of the tissue.

### BRIEF DESCRIPTION OF FIGURES

Non-limiting examples of embodiments of the present invention are described below with reference to figures attached hereto and listed below. In the figures, identical structures, elements or parts that appear in more than one figure are generally labeled with a same numeral in all the figures in which they appear. Dimensions of components and features shown in the figures are chosen for convenience and clarity of presentation and are not necessarily shown to scale.

FIGS. 1A and 1B schematically show a tissue viability monitor (TVM), diagnosing viability of tissue in an organ of a patient, in accordance with an embodiment of the present invention;

FIGS. 2A-2C schematically show the TVM shown in FIGS. 1A and 1B diagnosing tissue viability by determining a temperature relaxation time of the tissue, in accordance with an embodiment of the present invention;

FIGS. 3A-3B schematically show the TVM shown in FIGS. 1A and 1B diagnosing tissue viability in a patient's brain by determining a temperature relaxation time of the tissue, in accordance with an embodiment of the present invention;

FIG. 4 schematically shows a TVM useable for diagnosing tissue viability percutaneously, in accordance with an embodiment of the present invention; and

FIG. 5 schematically shows a probe end of a catheter for percutaneous viability diagnosis comprising a plurality of optical apertures for illuminating tissue being diagnosed for viability, in accordance with an embodiment of the present invention.

### DETAILED DESCRIPTION OF EXEMPLARY EMBODIMENTS

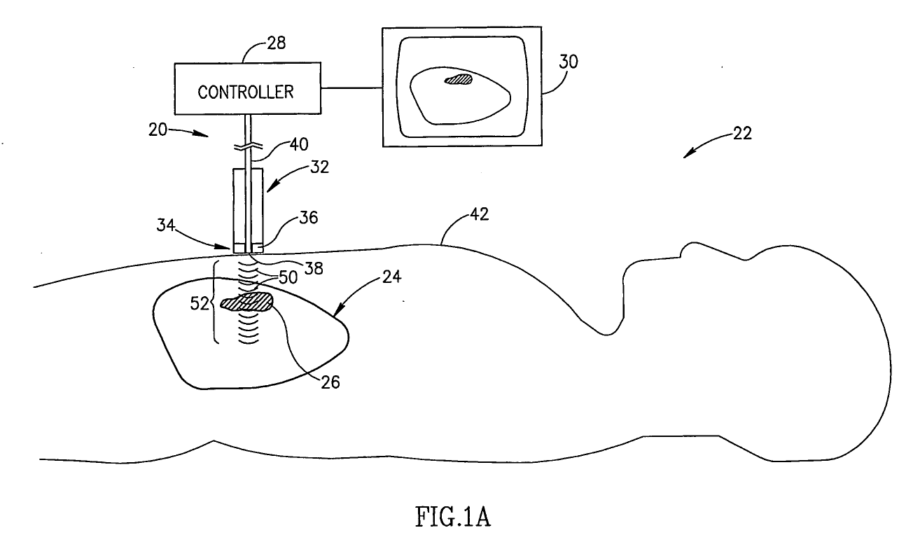

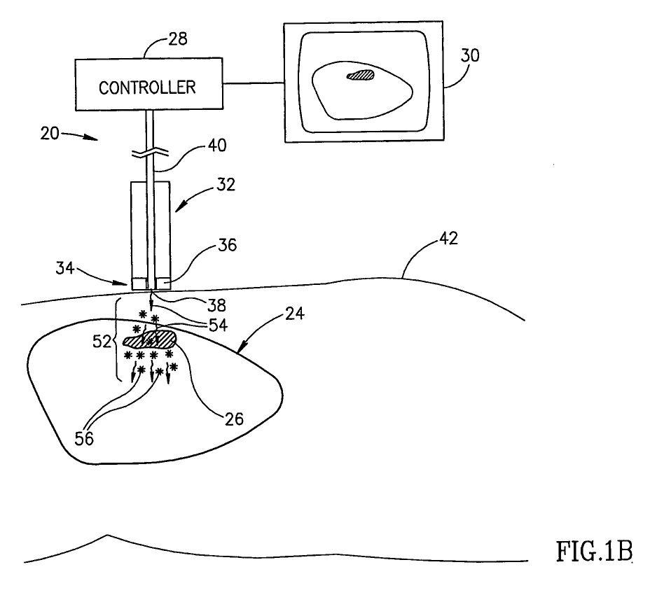

FIGS. 1A and 1B schematically show a TVM 20 being used to diagnose viability of tissue in an organ of a patient 22, in accordance with an embodiment of the present invention. By way of example, the organ is the liver 24 of the patient, which is shown in a cross-sectional view of the abdominal region of the patient. Liver 24, by way of example, has an ischemic region 26 of tissue having compromised viability. FIG. 1B shows a portion of the patient shown in FIG. 1A and features of TVM 20 greatly magnified for convenience of presentation.

TVM 20 optionally comprises a controller 28, a visual display console 30 and a wand 32 having a probe end 34. Wand 32 is shown in cross section. TVM 20 comprises at least one acoustic transducer 36, optionally located in probe end 34 of wand 32, and a light source that transmits light from an optical aperture 38 located in the probe end. By way of example, in TVM 20, aperture 38 is a first end of an optical fiber 40 having a second end (not shown) connected to a suitable light source (not shown) comprised in controller 28. At least one acoustic transducer 36 optionally comprises an annular acoustic detector that is formed with a hole in its center through which optic fiber 38 passes.

Configurations of acoustic detectors and light sources for the practice of the present invention other than that comprised in TVM 20 as shown in FIG. 1A and figures that follow will readily occur to a person of the art. For example, at least one acoustic transducer 36 may comprise an acoustic transducer located to one side of fiber 40 or a plurality of acoustic detectors configured in a circular array that surrounds fiber 40. In some embodiments of the present invention, at least one acoustic transducer 36 comprises a phased array of transducers. At least one transducer 36 may also comprise a transducer or array of transducers that are not located in wand 32 and are affixed to various appropriate locations on the skin of patient 22. Optical aperture 38 may be an optical aperture different from that shown in FIGS. 1A and 1B. For example, aperture 38 may be an aperture of a suitable laser or light-emitting-diode (LED) located in probe end 34 of wand 32. To diagnose tissue in liver 24 of patient 22 for viability, wand 32 is moved over skin 42 that overlays the patient's liver with probe end 34 of the wand in contact with the skin.

In some embodiments of the present invention, wand 32 is moved manually. In some embodiments of the present invention wand 32 is moved by a suitable apparatus. At each of a plurality of positions on skin 42, as probe end 34 is moved over the skin, controller 28 optionally controls acoustic transducer 36 to acquire an acoustic A-scan of tissue located below the position. As discussed below, controller 28 then controls the light source in the controller and transducer 36 to acquire data from which to determine viability of tissue located below the position. A-scans and viability determinations for the plurality of positions are used to provide a spatial map of tissue viability of liver 24, which is, optionally, displayed on console 30.

FIGS. 1A and 1B schematically illustrate performing viability diagnosis measurements at a given position on skin 42 during the viability scan of the patient's liver 24, in accordance with an embodiment of the present invention. In FIG. 1A controller 28 controls acoustic transducer 36 to transmit ultrasound, represented by curved lines 50, into the patient's body to generate an A-scan of a region 52 of tissue below the given position and an image liver 24. Following the A-scan of region 52, in FIG. 1B, controller 28 controls the light source to illuminate tissue in region 52 with at least one pulse of light, represented by wavy arrows 54, that is absorbed by an analyte whose concentration and/or change therein is useable as an indicator of viability. For example, the analyte may be any of the analytes, such as cytochrome a,a₃ or Hydrogen ions (as measured by pH) noted in the US Patents referenced above.

Energy absorbed from at least one light pulse 54 by the analyte in a given tissue voxel of region 52 generates photoacoustic waves that radiate out from the voxel. Photoacoustic waves generated in region 52 responsive to light 54 are represented by starbursts 56. A portion of the acoustic energy in photoacoustic waves 56 is incident on acoustic transducer 36, which generates signals responsive to the incident energy and transmits the signals via a suitable signal cable (not shown) to controller 28. Controller 28 processes the signals to determine locations in region 52 from which the acoustic energy arrives at transducer 36 and concentration of the analyte at the locations.

By way of example, in FIGS. 1A and 1B assume that viability is determined as a function of hemoglobin concentration in tissue of liver 24 as indicated, optionally by concentration of hemoglobin (Hb). In accordance with an embodiment of the present invention, region 52 might therefore be illuminated with light at a wavelength of about 810 nm, for which oxygenated and non-oxygenated hemoglobin have a substantially same absorption coefficient to stimulate photoacoustic waves in the region. Ischemic region 26 of liver 24 has poor circulation and therefore a low concentration of Hb. As a result, intensity of photoacoustic waves generated in ischemic region 26 responsive to light 54 is relatively low, which is schematically indicated in FIG. 1B by a relatively low concentration of starbursts 56 in the ischemic region.

Signals generated by acoustic transducer 36 responsive to acoustic energy incident on the transducer from photoacoustic waves 56 are transmitted to controller 28. The signals are processed and analyzed, “time resolved” as a function of time, using methods known in the art to determine concentration of Hb in region 52 as a function of location in the region. The result of the processing is an Hb concentration, viability “A-scan” of region 52, that indicates degree of viability as a function of a spatial coordinate in the direction along which region 52 is illuminated by light 54. Ischemic region 26 is identified and spatially located by signals indicating arrival at acoustic transducer 36 of relatively low intensity acoustic energy and a time of arrival of the energy following a time at which region 52 is illuminated with light 54. Concentration of Hb may be relative or absolute concentration.

In the above description, signals responsive to concentration of a “viability analyte” are generated responsive to characteristics of photoacoustic waves received from tissue voxels in liver 24. In some embodiments of the present invention, to assay a viability analyte, controller 28 controls acoustic transducer 36 to transmit ultrasound into region 52 during or after illumination of the region with light 54. At least one acoustic transducer 36 receives acoustic energy reflected from the transmitted ultrasound by tissue voxels illuminated with light 54 and generates signals responsive thereto. Controller 28 processes the reflected signals to determine effects of light 54 on optical or acoustic characteristics of the voxels and uses the determined effects to determine concentration of the analyte. Methods of determining the effects and using them to assay an analyte are described in PCT Publication WO 02/15776, referenced above.

It is noted, that in general, to determine concentration of an analyte in a region of biological tissue or a change in the concentration using the photoacoustic effect it is often necessary or advantageous to measure the photoacoustic effect at each of a plurality of wavelengths, wherein for at least one of the wavelengths the analyte absorbs light. For such situations, to determine viability responsive to concentration and/or change therein of a suitable analyte, controller 28 controls the light source to illuminate region 52 with at least one pulse of light at each of at least two appropriate different wavelengths. Signals generated by transducer 36 responsive to photoacoustic waves generated by the light are used, in accordance with an embodiment of the present invention, to determine viability of tissue in liver 24 as a function of location in the liver.

It is further noted that whereas in the above description TVM 20 images liver 24 and tissue in the abdomen of the patient using ultrasound transmitted by acoustic transducer 36, in some embodiments of the present invention controller 28 images the liver and abdominal tissue using the photoacoustic effect. Any suitable photoacoustic imaging method known in the art may be used in the practice of the present invention to image liver 24 and the abdominal tissue.

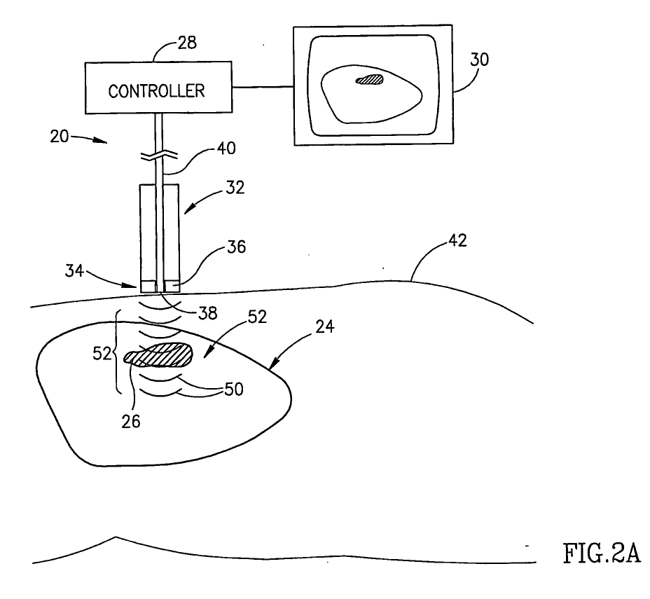

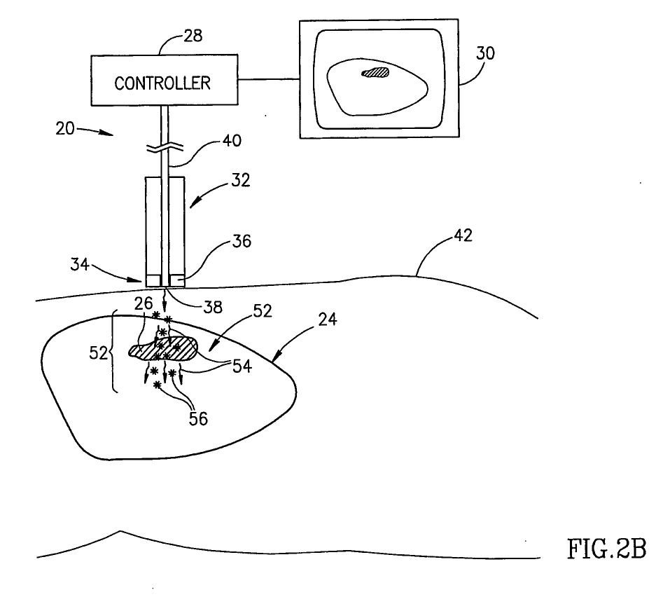

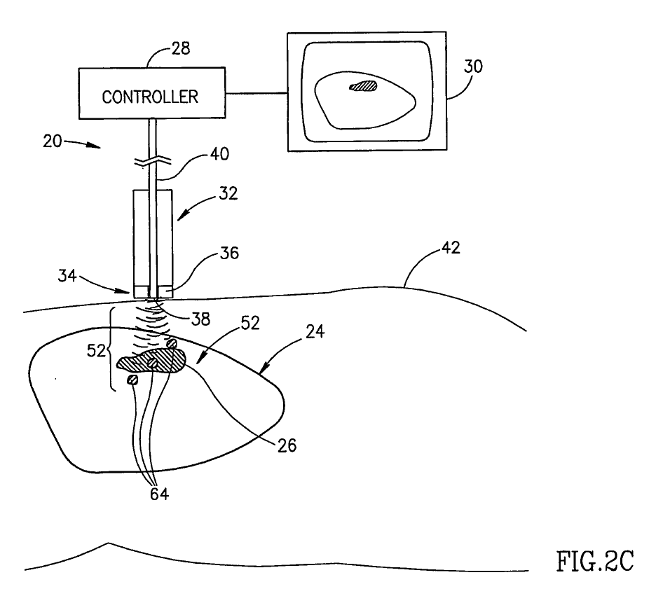

In some embodiments of the present invention, TVM 20 determines a temperature relaxation time of tissue in liver 24 to determine viability of tissue in the liver. To perform such viability measurements, acoustic transducer 36 comprises a phased array of acoustic transducers that is controllable to focus acoustic energy to relatively small regions of tissue in liver 24. FIGS. 2A-2C schematically show TVM 20 determining temperature relaxation times of tissue regions in liver 24 to diagnose viability, in accordance with an embodiment of the present invention.

In FIG. 2A as in FIG. 1A, probe end 34 is positioned on skin 42 over a region 52 of the abdomen of patient 22 and controller 28 controls transducer 36 to image tissue in the abdomen below probe end 34 and thereby tissue in a region of liver 24 with ultrasound. Thereafter, optionally, TVM 20 determines an ambient temperature of tissue in region 52. In accordance with an embodiment of the present invention, as schematically shown in FIG. 2B, temperature is determined by measuring the temperature of water in tissue in region 52 using the photoacoustic effect in accordance with a method described in U.S. Provisional Application 60/331,408 referenced above. Controller 28 controls the light source to illuminate region 52 with light represented by wavy arrows 54 at at least one wavelength at which light is absorbed by water to stimulate generation of photoacoustic waves represented by starbursts 56. Signals generated responsive to photoacoustic waves 56 generated by acoustic transducer 36 are processed by controller 28 to determine an absorption coefficient for water in region 52. The known dependence of the absorption coefficient of water on temperature at the at least one wavelength is used to determine the ambient temperature of region 52.

Thereafter, in FIG. 2C controller 28 controls transducer 36 to focus ultrasound on at least one tissue region in liver 24 to heat the region and raise its temperature by a desired amount above the ambient temperature of the liver. By way of example, in FIG. 2C controller 28 controls transducer 36 to focus ultrasound and heat each of a plurality of different tissue regions 64 in liver 24. Optionally, during heating of regions 64 temperatures of the regions are periodically determined and the determined temperatures used to monitor and control heating. Temperature of each region 64 is optionally measured similarly to the way in which ambient temperature of region 52 is measured, by photoacoustically measuring the temperature of water in the regions.

It is noted that the methods of photoacoustically measuring temperature described in U.S. Provisional Application 60/331,408 enable measuring temperature of a region comprising water as a function of location in the region. As a result the methods enable temperature of each of regions 64 to be determined, in accordance with an embodiment of the present invention, independently of temperature of the other regions.

In some embodiments of the present invention temperatures of regions 64 are measured by other, preferably non-invasive, techniques. For example, an article by R. Seif et. al. entitled “Estimation of Tissue Temperature Response to Heating Fields”, IEEE on Transactions of Biological Engineering, Vol. 42 No. 8, August 1995 pp 826-839, the disclosure of which is incorporated herein by reference, describes methods of measuring temperature of biological tissue that may be used in the practice of the present invention. The described methods use frequency shifts of ultrasound scattered from the tissue relative to a fundamental acoustic frequency of the structure of the tissue to determine temperature of the tissue.

Subsequent to heating tissue regions 64, controller 28 controls the light source and acoustic transducer 36 to photoacoustically determine temperatures of each of heated regions 64 at a plurality of different times as the temperature of the regions relax back to the ambient temperature. Controller 28 determines from the measured temperatures temperature relaxation times for the regions. The temperature relaxation times are used to assess viability of tissue in regions 64.

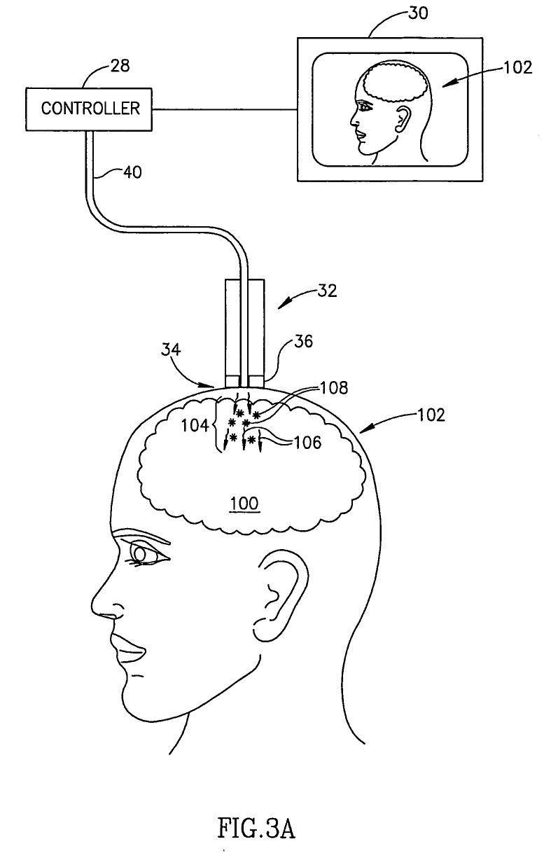

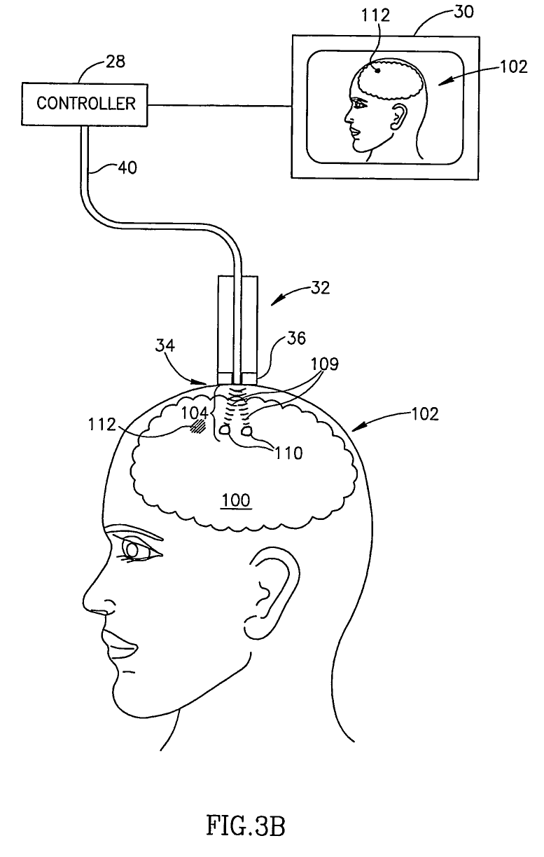

FIGS. 3A and 3B schematically show another example of TVM 20 determining tissue viability by determining temperature relaxation time of the tissue, in accordance with an embodiment of the present invention. In FIGS. 3A and 3B TVM 20 is schematically shown determining temperature relaxation times of tissue in the brain 100 of a patient 102 to diagnose viability of the tissue, in accordance with an embodiment of the present invention.

In FIG. 3A probe end 34 of wand 32 is positioned on the head of patient 102. The position of wand 32 relative to the patient's head is determined using any of various positioning methods and apparatus, such as for example methods and apparatus used to locate ultrasound scanners, known in the art.

After positioning of wand 32, TVM 20 determines an ambient temperature in a region 104 of the brain located beneath probe end 34, optionally by photoacoustically determining the temperature of water in the tissue. Controller 28 controls the light source to illuminate region 104 with light represented by wavy arrows 106 at at least one wavelength at which light is absorbed by water to stimulate generation of photoacoustic waves represented by starbursts 108. Signals generated responsive to photoacoustic waves 108 generated by acoustic transducer 36 are processed by controller 28 to determine an absorption coefficient for water in region 52 and therefrom temperature of region 104.

Thereafter, in FIG. 3B controller 28 controls transducer 36 to focus ultrasound 109 on tissue in at least one sub-region of region 110 of region 104 to heat the at least one sub-region and raise its temperature by a desired amount above the ambient temperature of the brain. By way of example, in FIG. 3B controller 28 controls transducer 36 to focus ultrasound and heat each of two different tissue sub-regions 110 in region 104. Optionally, during heating of sub-regions 110, temperatures of the regions are periodically determined, optionally by photoacoustically measuring the temperature of water in the regions, and the determined temperatures used to monitor and control heating.

Whereas in FIG. 3B sub-regions 110 that are heated by TVM 20 are sub-regions of region 104, sub-regions 110 are not necessarily sub-regions of region 104. Assuming that the ambient temperature of the brain is substantially the same for all regions of the brain, once an ambient temperature for brain tissue is determined, for example as described above by measuring temperature of region 104, sub-regions 110 do not have to be located within region 104.

Subsequent to heating tissue regions 110, controller 28 controls the light source and acoustic transducer 36 to optionally photoacoustically determine temperatures of each of heated sub-regions 110 at a plurality of different times as the temperature of the regions relax back to the ambient temperature. Controller 28 determines from the measured temperatures temperature relaxation times for the regions and therefrom viability of tissue in the regions.

The process is repeated as required for different sub-regions 110 of region 104 and for sub-regions in other parts of the brain of patient 102 to determine viability of tissue in the brain as a function of location and provide a viability map of the brain. Optionally, the viability map is displayed on console 30. By way of example, as a result of a stroke, patient 102 has damaged brain tissue in a region 112 which is diagnosed, in accordance with an embodiment of the present invention, as having impaired viability and which is displayed on console 30.

It is noted that in a TVM, in accordance with an embodiment of the present invention, similar to TVM 20, the at least one acoustic transducer is comprised in a wand, which is moved over the skin of the body. In some TVMs, in accordance with an embodiment of the present invention, the at least one transducer comprises at least one transducer that is attached to the skin at a fixed location and not moved during viability diagnosis of tissue in the body. In some embodiments of the present invention, the at least one transducer comprises an array of transducers, such as a phased array affixed to the skin.

A TVM, in accordance with an embodiment of the present invention, may also comprise more than one optical aperture through which light is transmitted to tissue in the body to stimulate a photoacoustic effect in the body. A TVM, in accordance with an embodiment of the present invention, comprising a plurality of optical apertures for illuminating tissue being diagnosed for viability can simultaneously acquire data for a plurality of viability A-scans of the region. In some embodiments of the present invention, a TVM comprises an optical aperture that is not mounted in a wand, which is moved over the skin during viability diagnosis, but comprises at least one optical aperture positioned at a fixed location on the skin during viability diagnosis.

In some embodiments of the present invention, components of a TVM are mounted in a catheter suitable for percutaneous introduction into a patient's body to diagnose viability of tissue in an organ of the patient. Percutaneous viability diagnosis can be advantageous for performance of various different therapies, such as for example performance of percutaneous myocardial revascularization. Methods for performance of percutaneous myocardial revascularization are described in a PCT application entitled “Methods and Apparatus for Performing Myocardial Revascularization” filed on even date with the present application, the disclosure of which is incorporated herein be reference.

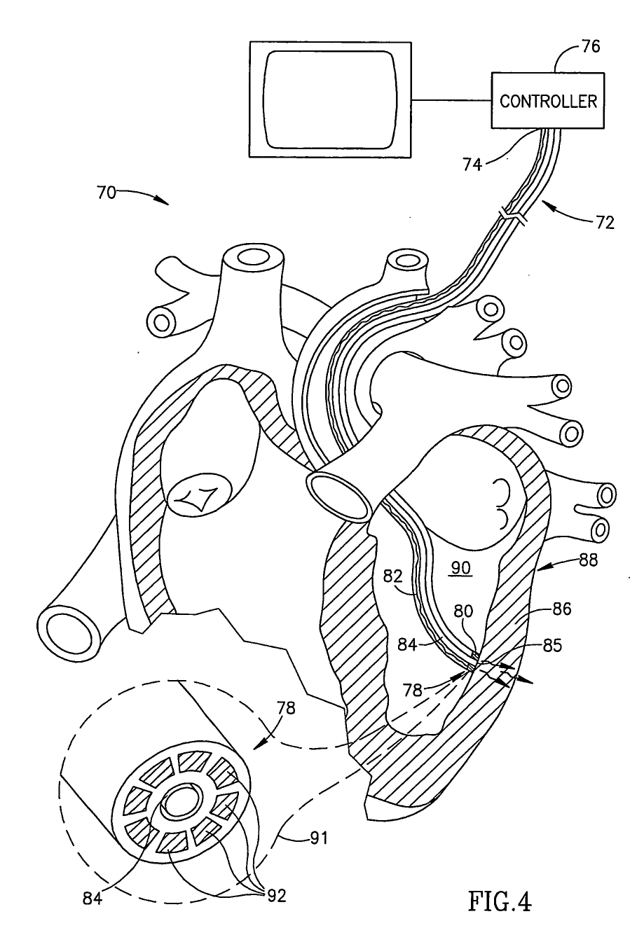

FIG. 4 schematically shows a “percutaneous” TVM 70, in accordance with an embodiment of the present invention, comprising a catheter 72 having a control end 74 coupled to a controller 76 and a probe end 78 in which at least one acoustic transducer 80 is mounted. Signals are transmitted to and/or from at least one acoustic transducer 80 via a suitable signal cable 82 in catheter 72. At least one optic fiber 84 extends the length of catheter 72 and transmits light from a suitable light source (not shown) in controller 76 to an end 85 (i.e. an optical aperture) of the fiber in probe end 78. Light is transmitted from end 85 to illuminate tissue being diagnosed for viability. In operation, catheter 72 is threaded through the patient's vascular system to position probe end 78 close to or contiguous with tissue to be tested for viability. In FIG. 4, by way of example, TVM 70 is shown diagnosing viability of a region 86 of tissue in the heart wall 88 of the left ventricle 90 of a patient's heart.

At least one acoustic transducer 80 may have any appropriate form and configuration known in the art. In some embodiments of the present invention, at least one transducer 80 comprises a plurality of transducers. In some embodiments of the present invention, the plurality of transducers is controlled by controller 76 to operate as a phased array. An exemplary configuration of at least one acoustic transducer 80 and optic fiber 84 in probe end 78 is shown greatly magnified in inset 91. In the inset, at least one acoustic transducer 80 comprises an array of acoustic transducers 92 that controller 76 optionally operates as a phased array.

TVM 70 operates similarly to TVM 20 and tests viability of region 86 by illuminating the region with at least one pulse of light at a suitable wavelength to stimulate generation of photoacoustic waves in the region. Transducer 80 generates signals responsive to the photoacoustic waves, which are transmitted via signal cable 82 to controller 76. Controller 76 processes the signals to assess tissue viability.

In some embodiments of the present invention, the signals are processed to provide a measure of viability responsive to concentration of and/or change in concentration of a suitable viability analyte. In some embodiments of the present invention, controller 76 controls acoustic transducer 80 to focus ultrasound on region 86 and heat the region to a temperature above an ambient temperature of tissue in heart wall 88. (For embodiments of the present invention in which ultrasound is focused to heat tissue, at least one acoustic transducer 80 comprises a phased array of transducers or a focused transducer.) Photoacoustic signals subsequently generated by acoustic transducer 80 are used by controller 76 to repeatedly measure temperature of region 86 as the temperature relaxes to the ambient temperature. The measurements are used to determine a temperature relaxation time for region 86. The temperature relaxation time is used to assess blood flow in the region and therefrom viability of region.

In some embodiments of the present invention a TVM similar to TVM 70 is used to perform therapy on a region that it diagnoses for viability. For example, controller 76 in TVM 70, following assessment of viability of region 86, if the region is diagnosed as ischemic, optionally controls the light source to transmit light from end 85 of fiber 82 that ablates tissue in the region to form a hole therein and stimulate revascularization.

In general a TVM, in accordance with an embodiment of the present invention, having a single optical aperture from which light is transmitted to illuminate a region of tissue being diagnosed for viability acquires data for a single viability A-scan of the region at any given time. In the A-scan, tissue viability is provided as a function of a single spatial coordinate along a direction in which the tissue is illuminated with light from the aperture. To provide a three-dimensional map of viability of tissue in a region of an organ the aperture is moved to scan the region and acquire a plurality of A-scans of the region. For example, to provide a three dimensional viability map of heart wall 88 shown in FIG. 4, probe end 78 of catheter 72 is moved to scan the heart wall and acquire data for a plurality of A-scans of the heart wall.

In some embodiments of the present invention, a TVM comprising a catheter for percutaneous viability diagnoses comprises a plurality of optical apertures in the probe end of the catheter. For a fixed position of the probe end in a neighborhood of a region of tissue, light transmitted form each of the optical apertures illuminates the region along a different direction. Such a percutaneous TVM can provide A-scan viability measurements along a plurality of different directions in the tissue region for a single position of the probe end of the catheter.

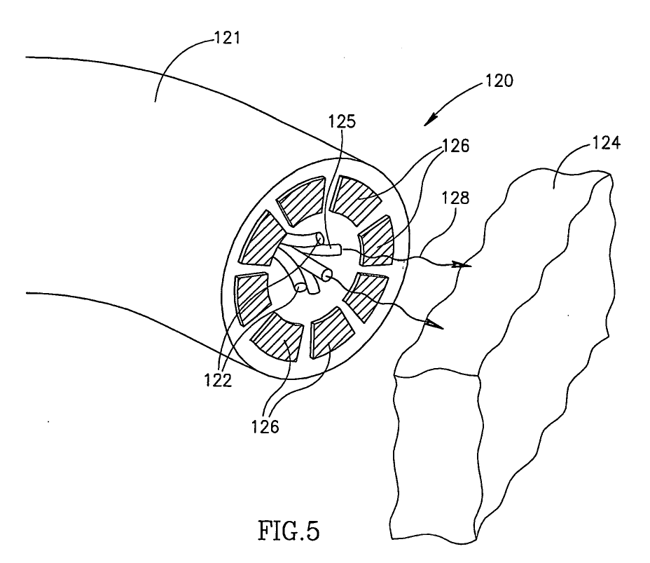

FIG. 5 schematically shows a magnified perspective view of a probe end 120 of a catheter 121 comprised in a percutaneous TVM (not shown), in accordance with an embodiment of the present invention, which probe end comprises a plurality of optical apertures 122. Optical apertures 122 enable a region of tissue 124 being diagnosed for viability using the TVM to be illuminated by light along a plurality of different directions. Each optical aperture 122 is optionally an end of an optic fiber 125 and probe end 120 optionally comprises a phased array of acoustic transducers 126. In operation, a controller in the TVM controls a suitable light source or sources to transmit light at a desired wavelength, optionally sequentially, from each fiber end 122. For each direction along which region 124 is illuminated by light from an aperture 122, signals generated by transducers 126 responsive to photoacoustic waves generated by the light are processed to provide a viability A-scan of tissue for the direction. FIG. 5 schematically shows region 124 being illuminated by light represented by wavy arrows 128 transmitted from two of apertures 122.

In the description and claims of the present application, each of the verbs, “comprise” “include” and “have”, and conjugates thereof, are used to indicate that the object or objects of the verb are not necessarily a complete listing of members, components, elements or parts of the subject or subjects of the verb.

The present invention has been described using detailed descriptions of embodiments thereof that are provided by way of example and are not intended to limit the scope of the invention. The described embodiments comprise different features, not all of which are required in all embodiments of the invention. Some embodiments of the present invention utilize only some of the features or possible combinations of the features. Variations of embodiments of the present invention that are described and embodiments of the present invention comprising different combinations of features noted in the described embodiments will occur to persons of the art. The scope of the invention is limited only by the following claims.

1. A tissue viability monitor (TVM) for determining viability of a biological tissue comprising:
at least one light source controllable to illuminate the tissue with light that is absorbed by an analyte in the tissue to generate photoacoustic waves therein;
at least one acoustic transducer that generates signals responsive to the photoacoustic waves;
means for generating a temperature difference between temperature of the tissue and an ambient temperature of surrounding tissue; and
a controller adapted to control the means for generating a temperature difference in the tissue and to control the light source to illuminate the tissue with light absorbed by at least one analyte in the tissue and wherein the controller processes the signals generated by the at least one transducer to determine concentration of at least one analyte in the tissue and to determine temperature in the tissue and therefrom a relaxation time during which the temperature difference relaxes to zero and uses the concentration and relaxation time to provide a measure of viability.

2. A TVM in accordance with claim 1 wherein the controller processes the signals to determine locations of sources of the photoacoustic waves within the tissue.

3. A TVM in accordance with claim 2 wherein the locations of sources of photoacoustic waves are determined with a resolution equal to or better than about 100 micrometers.

4. A TVM in accordance with claim 2 wherein the locations of sources of photoacoustic waves are determined with a resolution equal to or better than about 50 micrometers.

5. A TVM in accordance with claim 2 wherein the locations of sources of photoacoustic waves are determined with a resolution equal to or better than about 20 micrometers.

6. A TVM in accordance with claim 1 wherein the at least one analyte is a plurality of analytes.

7. A TVM in accordance claim 1 wherein the at least one analyte comprises the redox state cytochrome a,a₃.

8. A TVM in accordance claim 1 wherein the at least one analyte comprises Hydrogen ions.

9. A TVM in accordance with claim 1 wherein the at least one analyte comprises hemoglobin.

10. A TVM in accordance with claim 1 wherein the at least one analyte comprises oxygenated hemoglobin.

11. A TVM in accordance with claim 1 wherein the means for generating a temperature difference comprises an acoustic transducer, which the controller controls to transmit acoustic waves to the tissue that generate the temperature difference.

12. A TVM in accordance with claim 1 wherein the controller determines temperature of the tissue during generation of the temperature difference to monitor the generation of the temperature difference.

13. A TVM in accordance with claim 12 wherein the controller controls the means for generating a temperature difference responsive to the determined temperature.

14. A TVM in accordance with claim 1 wherein to determine the relaxation time the light source illuminates the tissue with light at a wavelength at which light is absorbed by water to generate photoacoustic waves in the tissue and the controller uses the signals generated by the at least one transducer to determine temperature of water in the tissue and thereby of the tissue.

15. A TVM according to claim 1 and comprising a catheter having a probe end that is positioned in a neighborhood of or in contact with the tissue to determine tissue viability and wherein the light source comprises an optic fiber having an optic end located at the probe end from which optic end light that illuminates the tissue is radiated.

16. A TVM in accordance with claim 15 wherein the at least one acoustic transducer comprises at least one acoustic transducer mounted in the probe end of the catheter.

17. A tissue viability monitor (TVM) for determining viability of a biological tissue comprising:
at least one light source controllable to illuminate the tissue with light that is absorbed by an analyte in the tissue to generate photoacoustic waves therein;
at least one transmitting acoustic transducer controllable to transmit waves that are incident on the tissue;
at least one sensing acoustic transducer that generates signals responsive to the photoacoustic waves and waves from the incident waves that are reflected by the tissue;
means for generating a temperature difference between temperature of the tissue and an ambient temperature of surrounding tissue; and
a controller that processes the signals responsive to photoacoustic waves to determine concentration of at least one analyte in the tissue and the signals responsive to reflected waves to determine temperature of the tissue and therefrom a relaxation time during which the temperature difference relaxes to zero and wherein the controller uses the concentration and relaxation time to provide a measure of viability.

18. A TVM according to claim 17 wherein the characteristic is a frequency shift of the scattered acoustic waves relative to a fundamental acoustic frequency of the structure of the tissue.

19. A method of determining viability of a biological tissue comprising:
generating a temperature difference between temperature of the tissue and an ambient temperature of surrounding tissue;
illuminating the tissue with light that is absorbed by at least one analyte in the tissue to generate photoacoustic waves therein;
determining concentration of an analyte in the tissue responsive to the photoacoustic waves;
determining a relaxation time during which the temperature difference relaxes to zero responsive to the photoacoustic waves; and
providing a measure of viability responsive to the concentration and the relaxation time.

20. A method of determining viability of a biological tissue comprising:
illuminating the tissue with light that is absorbed by at least one analyte in the tissue to generate photoacoustic waves therein;
determining concentration of at least one analyte in the tissue responsive to the photoacoustic waves;
generating a temperature difference between temperature of the tissue and an ambient temperature of surrounding tissue;
transmitting acoustic waves that are incident on and reflected by the tissue;
determining a relaxation time during which the temperature difference relaxes to zero responsive to the reflected acoustic waves; and
providing a measure of viability responsive to the concentration and the relaxation time.
